(19) United States
(12) Plant Patent Application Publication
(10) Pub. No.: US 2010/0251441 P1
(43) Pub. Date: Sep. 30, 2010

(54) Thornless honeylocust named 'Draves'

(50) ;

(76) Inventors: Timothy C. Brotzman, Madison, OH (US); Thomas M. Draves, Darien, NY (US)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/383,921

(22) Filed: Mar. 30, 2009

(51) Int. Cl. A01H 5/00

(52) U.S. Cl. PLT/217

(57) ABSTRACT

A thornless honeylocust tree named ‘Draves’, a medium tree distinguished by its strongly upright, columnar form.

### LATIN NAME OF THE GENUS AND SPECIES INCLUDING THE VARIETY DENOMINATION OF THE PLANT CLAIMED

The plant claimed relates to a new and distinct variety of thornless honeylocust, botanically known as Gleditsia triacanthos var. inermis, and known by the cultivar name Draves.

This new cultivar was found growing on a cultivated, maintained residential property near Darien, N.Y. This new cultivar was noted as distinctive because of its strongly upright, columnar form.

The Draves variety is the only variant of thornless honeylocust we are aware of that displays this strongly columnar form differing from the following broadly pyramidal selections of honeylocust: Fairview 50′×40′ (15.2 m×12.2 m), Imperial 25′×24′ (7.6 m×7.3 m) @15 yrs, Moraine 32′×28′ (9.8 m×8.5 m) @15 yrs, Perfection 50′×30′ (15.2 m×9.1 m), Shademaster 45′×35′ (13.7 m×10.7 m), Skyline 45′×35′ (13.7 m×10.7 m), True Shade 40′×30′ (12.2 m×9.1 m) having height to width ratios as noted (Dirr, Manual of Woody Landscape Plants, 4^{th} edit., 1990).

The original Draves cultivar, which is approximately 40 years old, is approximately 45′ tall and 18′ wide. Trunk diameter is approximately 13.5″ at breast height. The crown/branching is full from bottom to top with moderate taper. No thorns/spines have been observed. Seed pods have been observed. Tree form is strongly excurrent with the leader outgrowing the lateral branches, producing a strongly columnar shape with upright-growing lateral branches and a clearly defined central leader. Compound leaves are dark, blue-green and lustrous, primarily bipinnately compound in character on vigorous growth. Otherwise they are pinnate.

The Draves cultivar has been asexually reproduced at Brotzman's Nursery in Madison, Ohio by budding and grafting, and plants propagated by this method have displayed the same unique characteristics of the original plant.

Referring now to the accompanying drawings:

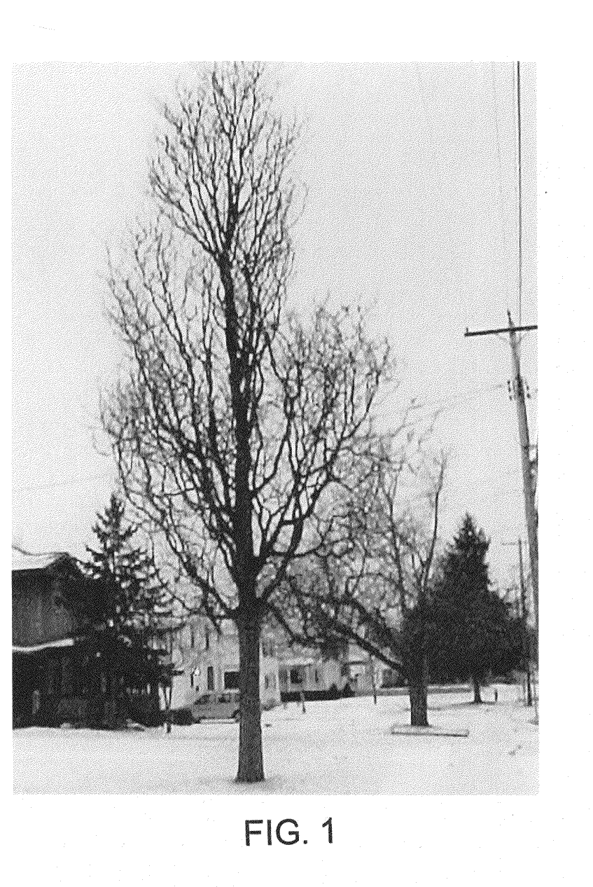

FIG. 1 is a colored photograph illustrating the overall appearance of the original cultivar Draves in winter without leaves.

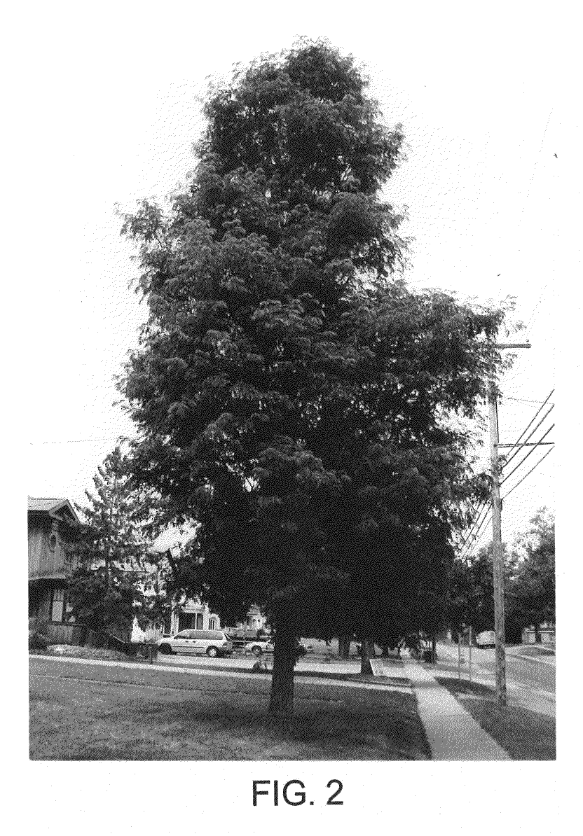

FIG. 2 is a colored photograph illustrating the overall appearance of the original Draves in the summer.

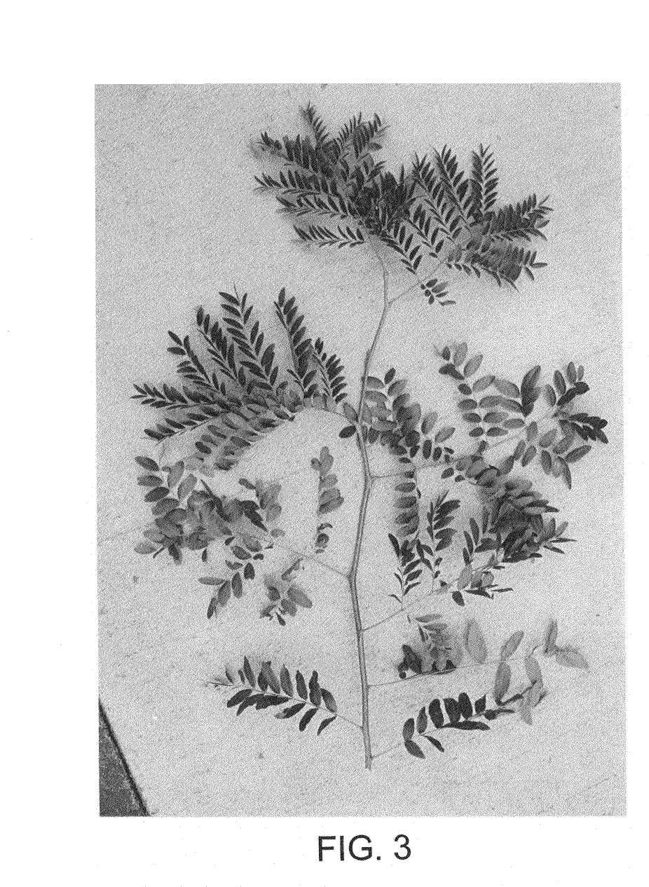

FIG. 3 is a colored photograph illustrating the new growth of Draves in the summer.

All of the drawing figures show the colors as truly as is reasonably possible to obtain in colored reproductions of this type.

The following is a detailed description of my new cultivar, Draves. In all cases, where color is different from the typical and is considered a distinguishing feature of this variety, reference is made to specific colors on the R.H.S. Colour Chart (1995 ed.) published by The Royal Horticultural Society, London, England. Here follows a detailed description of the characteristics of this cultivar, as displayed by specimens grown at Madison, Lake County, Ohio and the original tree near Darien, N.Y. Coloration of leaves and bark may be variable, due to conditions of nutrition, stress, age of plant, location on plant and the presence/absence of sun/shade. Plant part comparisons have been made using samples taken from a mature plant where growth rates and characteristics are considered typical.
Botanical description:
   Parentage.—Unknown — discovered growing on a cultivated, maintained residential property near Darien, N.Y.
   Hardiness.—Hardy in USDA Hardiness Zone 4.
   Growth rate.—Moderate, more rapid in youth.
   Form.—Strongly excurrent with the leader outgrowing the lateral branches, producing a strongly columnar shape with upright-growing lateral branches and a clearly defined central leader.
   Habit.—Central leader with densely arranged, lateral branches which display an upright orientation. Primary branches on the lower third of the trunk form an angle of approximately 45° with the trunk while those on the upper two-thirds of the trunk form angles of approximately 30°. Within 1.5′ to 3.0′ of the trunk, all of the primary branches are oriented such that they form an angle of approximately 30° with the trunk, which contributes to superior durability against winter storm damage.
   Bark.—Mature bark dark brown, rough.
   Leaves.—Pinnately compound with 8 to 20 oblong-lanceolate leaflets along the central axis, or bipinnately compound with 8 to 10 branches along a central rachis, each with 18-20 leaflets. Bipinnate leaves are most common on vigorous branches. Leaflets are 20 mm-30 mm long by 7 mm-12 mm wide, and are lustrous dark blue-green Yellow Green Group 147A. Autumn color is yellow, Yellow Group 12A. The base of the petiole is swollen and encloses the lateral bud.
   Twigs.—Show typical sympodial branching. New growth bright green Yellow Green Group 146A. Second year wood moderate to heavily lenticeled, darkening to Yellow Green Group 148A and Grey-Brown Group 199 A on older branches until formation of thicker, dark brown bark.
   Flowers.—Not observed.
   Seeds.—Generally considered male, but some seed pods have been observed.
   Spines.—Not observed.

1. A thornless honeylocust tree named ‘Draves’, as described and illustrated, a medium tree distinguished by its strongly upright, columnar form.
